(19) United States
(12) Patent Application Publication
(10) Pub. No.: US 2004/0173210 A1
(43) Pub. Date: Sep. 9, 2004

(54) Switching apparatus and method for oxygen delivery system

(76) Inventor: Lucky Campbell, Phoenix, AZ (US)

Correspondence Address: WEISS & MOY PC, 4204 NORTH BROWN AVENUE, SCOTTSDALE, AZ, 85251, US

(21) Appl. No.: 10/758,378

(22) Filed: Jan. 15, 2004

Related U.S. Application Data
(60) Provisional application No. 60/451,688, filed on Mar. 4, 2003.

(51) Int. Cl. A61M 9/00

(52) U.S. Cl. 128/202.24

(57) ABSTRACT

An oxygen switching apparatus and method switches a flow of oxygen under pressure as between a first oxygen carrying tube and a second oxygen carrying tube. In this manner, the flow of oxygen under pressure is restricted to one nostril at a time, and the switching can prevent one nostril from becoming unduly chafed or harmed by a continuous flow of oxygen thereto. The switch can be located within the housing containing the source of pressurized oxygen, or can be external thereto.

### RELATED APPLICATION

This non-provisional application claims priority from provisional application No. 60/451,688, filed on Mar. 4, 2003.

### FIELD OF THE INVENTION

This invention relates generally to oxygen delivery systems and, more particularly, to a switching apparatus and method for an oxygen delivery system that permits the switching of oxygen delivery between two nostrils.

### BACKGROUND OF THE INVENTION

It is often necessary to supply oxygen or a gas mixture to an individual requiring supplemental assistance with breathing. For persons who have such a need and who are ambulatory, a personal oxygen system is often utilized. Such a system permits a person to carry oxygen with him or her, meeting the person's oxygen needs while affording freedom of movement.

Typically, oxygen is administered from a system via a nasal cannula, comprising flexible supply tubing terminating in a cannula having a pair of nipple ends that are inserted in the nostrils of the recipient.

The HELIOS® personal oxygen system is an example of a personal oxygen system. It has an oxygen-containing housing that is small and lightweight, and provides a flow of oxygen for about 8-10 hours before refilling is necessary. In the HELIOS system, there are two tubes that exit, the housing, and that end in the recipient's nostrils. One tube carries oxygen to the user on demand in response to a pressure change sensed by the other tube. The second tube is the pressure sensing tube, and does not itself deliver oxygen to the recipient.

However, there is a drawback with this design. Because all of the supplemental oxygen is directed exclusively to one nostril, that nostril can become chafed, leading to patient discomfort. A need therefore exists for a switching apparatus and method that permits the switching of oxygen delivery back and forth between the two nostrils, while still permitting pressure sensing to occur.

### SUMMARY OF THE INVENTION

In accordance with one embodiment of the present invention, an oxygen switching apparatus is disclosed. It comprises, in combination: a first tube having a first end and a second end; a second tube having a first end and a second end; wherein the first end of the tube receives oxygen under pressure; a third tube having a first end and a second end; a fourth tube having a first end and a second end; a switch in communication with each of the second end of the first tube, the second end of the second tube, the first end of the third tube and the first end of the fourth tube; wherein the second end of the third tube terminates proximate a first nostril of a person; wherein the second end of the fourth tube terminates proximate a second nostril of the person; wherein the switch is positioned so as to permit a user, upon activation of the switch, to alternate a flow of the oxygen under pressure from the second end of the first tube, as between the first end of the third tube and the first end of the second tube so that the oxygen under pressure exits only one of the second end of the third tube and the second end of the fourth tube.

In accordance with another embodiment of the present invention, an oxygen switching apparatus is disclosed. It comprises, in combination: a source of oxygen under pressure; a single tube having a first end coupled to the source of oxygen under pressure and a second end; a second tube having a first end and a second end terminating proximate a first nostril of a person; a third tube having a first end and a second end terminating proximate a second nostril of a person; a switch in communication with and interposed between the second end of the first tube and the first end of each of the second tube and the first end of the third tube; the switch being positioned so as to permit a user, upon activation of the switch, to alternate a flow of the oxygen under pressure from the second end of the first tube as between the first end of the second tube and the first end of the third tube so that the oxygen under pressure exits only one of the second end of the second tube and the second end of the third tube.

In accordance with a further embodiment of the present invention, a method for switching delivery of oxygen between two oxygen carrying tubes is disclosed. The method comprises the steps of: providing a first tube having a first end and a second end; providing a second tube having a first end and a second end; wherein the first end of the tube receives oxygen under pressure; providing a third tube having a first end and a second end; providing a fourth tube having a first end and a second end; providing a switch in communication with each of the second end of the first tube, the second end of the second tube, the first end of the third tube and the first end of the fourth tube; wherein the second end of the third tube terminates proximate a first nostril of a person; wherein the second end of the fourth tube terminates proximate a second nostril of the person; wherein the switch is positioned so as to permit a user, upon activation of the switch, to alternate a flow of the oxygen under pressure from the second end of the first tube, as between the first end of the third tube and the first end of the second tube so that the oxygen under pressure exits only one of the second end of the third tube and the second end of the fourth tube; receiving oxygen under pressure in the first end of the first tube; and activating the switch and thereby causing the oxygen under pressure from the second end of the first tube to be received in the other of the first end of the third tube and the first end of the fourth tube.

### BRIEF DESCRIPTION OF THE DRAWINGS

FIG. 1 is a perspective view of an embodiment of an oxygen delivery system incorporating an embodiment of a switch consistent with the present invention.

FIG. 1A is a perspective view of the oxygen-containing housing portion of an oxygen delivery system, having an integral switch consistent with the present invention.

FIG. 2 is a side, cross-sectional view of the switch shown in FIG. 1, taken along line 2-2.

FIG. 3 is a side view of the switch shown in FIG. 1, and further illustrating the flow between the oxygen containing housing and the switch, and the flow between the switch and the recipient.

FIG. 4 is a front view of the nipple assembly component of the oxygen delivery system of FIG. 1.

FIG. 5 is a perspective view of another embodiment of an oxygen delivery system, having an integral switch consistent with the present invention.

### DETAILED DESCRIPTION OF THE PREFERRED EMBODIMENTS

The method of the present invention can be accomplished in a number of different ways. In one embodiment, a switch 10 is positioned outside of an oxygen-containing housing 12. See, e.g., FIG. 1, 3 and 5. In another embodiment, a switch (not shown) may be positioned within the oxygen-containing housing 12. See FIG. 1A. These will now be discussed in more detail.

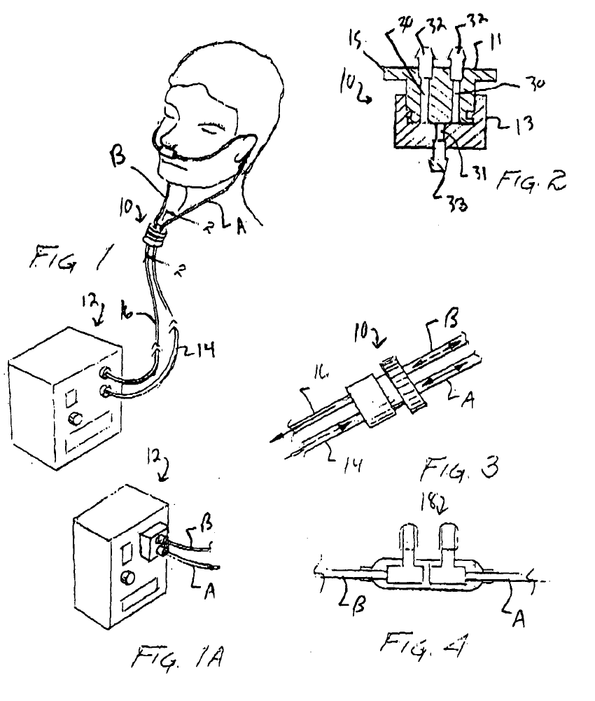

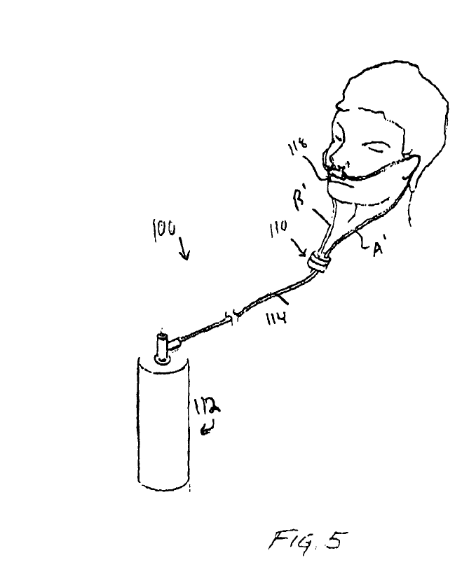

Turning first to the external switch 10 and referring first to FIGS. 1, 3 and 5, an output (oxygen-delivering) tube 14 and an input (pressure-sensing) tube 16 are fed into a first end of the switch 10. Two tubes (A and B) exit a second end of the switch 10. By activation of the switch 10, oxygen can be alternately delivered from tube 14, through switch 10, to tube A or tube B, with pressure sensing operating through the other of tube A or tube B. Pressure sensing will always, in this embodiment, occur via tube 16 below switch 10, and will alternate between tube A and tube B depending on the position of the switch 10.

It will be preferred that the switch 10 be of a type that is highly visible to the user, so that it will be clear to the user which tube is carrying oxygen and which is sensing pressure. For example, color coding could be provided, so that a dot or other marking on the switch 10 would align with a corresponding symbol on one of the tubes A and B. In this way, a simple glance will immediately tell the user (or health care provider) which tube is carrying oxygen.

It will further be preferred that the switch 10 be of a type that is positive in nature; i.e., that is either in one condition or another, but is not susceptible of being left in a middle position where oxygen is being simultaneously delivered to both tubes A and B, which would interfere with the pressure sensing ability of the system. The switch 10 could be mechanical, electrical or pneumatic (perhaps utilizing air from the oxygen system itself).

Referring specifically to FIG. 2, an example of a mechanical version of the switch 10 is shown by way of example, and not by way of limitation. In this embodiment, the switch 10 comprises a superior section 11 and an inferior section 13. The superior section 11 has two channels 30 therethrough, each with a cannula coupling member 32 positioned at the superior side thereof. Tubes A and B will each be coupled at an open end thereof to a coupling member 32. (Of course, it should be noted that other means for coupling tubes A and B to the switch 10 could be provided, and coupling members 32 are shown only by way of example.) The inferior section 13 has two channels 31 therethrough (only one of which is visible in FIG. 2), each with a cannula coupling member 33 positioned at the superior side thereof. Tubes A and B will each be coupled at an open end thereof to a coupling member 32. In the embodiment of FIG. 5, discussed below, it will be necessary to provide only a single channel 30 and corresponding coupling member 32 in the inferior section 13.

During operation, air will flow through tube 14, through channel 31, through channel 30 which is continuous with channel 31, and will enter tube A. Tubes B and 16 will also be in communication through channels 30 and 31, permitting pressure sensing at the housing 12.

Still referring to FIG. 2, the superior section 11 may be rotated relative to the inferior section 13 by grasping the outer rim 15 of the superior section 11, and rotating the superior section 11 180 degrees. This will have the affect of placing Tubes 14 and B in communication, and Tubes 16 and A in communication—i.e., the reverse of the prior configuration. As a result, pressure sensing will now occur through Tube A, and air will be delivered through Tube B (though the oxygen carrying and pressure sensing roles of tubes 14 and 16 will not change).

A person using an oxygen system having a switching system as above-described can operate it in a number of different ways. The person could wait until chafing or discomfort first develops in one nostril, and then switch oxygen delivery to the second nostril. Alternatively, the person could regularly switch oxygen delivery back and forth, perhaps according to a fixed schedule (e.g., allowing two hours of oxygen delivery to one nostril and then switching to the other for the next two hours).

It should be apparent that a timing device could be utilized as well, with the timing device preferably being adjustable to take into account the sensibilities and needs of different patients. In addition, the timing device could simply be of an alarm-type, notifying the user that the specified period of time has passed so that the user can then operate the switch. Or, the timing device could be coupled to the switching mechanism, automatically switching oxygen delivery from tube A to tube B at prescribed intervals.

Referring now to FIG. 1A, in another embodiment, the switch (not shown) is located within the oxygen-containing housing 12. In this embodiment, the output (oxygen-delivering) and input (pressure-sensing) tubes (not shown) are fed into a first end of a switch that is also located within the housing 12. Two tubes (A and B) exit a second end of the switch and thereafter the housing 12, with the result that the switch is not exposed.

In this embodiment, operation of the switch should be through controls that are located on the oxygen-containing housing 12. Again, as described above, operation of the switch can be manual, or can be automatic according to a pre-set schedule.

It should be apparent that one advantage of the methods herein described is that they permit the switching of oxygen delivery from one tube to the other without requiring the patient to actually remove the nipple ends from the nostrils. FIG. 4 illustrates the nipple ends 18, which are coupled to tubes A and B and are inserted into the user's nostrils. Actual removal and reinsertion of the nipple ends 18 can itself be uncomfortable, and creates an infection risk.

It should be noted that the method of the present invention could be utilized with other oxygen-delivery systems, including ones that do not utilize the pressure-sensing tube of the HELIOS® system. Referring now to FIG. 5, a system 100 is shown, in which oxygen from oxygen source 112 flows through a single tube 114. Coupled to the tube 114 is a switch 110. Tubes A and B have nipple ends 118 that are inserted into the nostrils.

But for the switch 110, both nostrils would simultaneously receive the delivery of oxygen. However, even where oxygen is simultaneously being delivered to both nostrils rather than solely to one, chafing and discomfort are possibilities. Operation of the switch 110 causes one of tube A or tube B to receive the flow of oxygen, with the other tube being sealed. This allows the user to give each nostril a period rest from the receipt of pressurized gas. As shown in FIG. 5, the switch 110 could be external to the oxygen source 1112. Alternatively, the switch could be placed internally to the oxygen source 12, in which case the two exit tubes would be required to exit the housing.

While the invention has been particularly shown and described with reference to the preferred embodiments thereof, it will be understood by those skilled in the art that the foregoing and other changes in form, and details may be made therein without departing from the spirit and scope of the invention.

I claim:

1. An oxygen switching apparatus comprising, in combination:
a first tube having a first end and a second end;
a second tube having a first end and a second end;
wherein said first end of said tube receives oxygen under pressure;
a third tube having a first end and a second end;
a fourth tube having a first end and a second end;
a switch in communication with each of said second end of said first tube, said second end of said second tube, said first end of said third tube and said first end of said fourth tube;
wherein said second end of said third tube terminates proximate a first nostril of a person;
wherein said second end of said fourth tube terminates proximate a second nostril of said person;
wherein said switch is positioned so as to permit a user, upon activation of said switch, to alternate a flow of said oxygen under pressure from said second end of said first tube, as between said first end of said third tube and said first end of said second tube so that said oxygen under pressure exits only one of said second end of said third tube and said second end of said fourth tube.

2. The oxygen switching apparatus of claim 1 further comprising a source of oxygen under pressure within a housing and wherein said switch is coupled to said first tube and said second tube in an interior of said housing.

3. An oxygen switching apparatus comprising, in combination:
a source of oxygen under pressure;
a single tube having a first end coupled to said source of oxygen under pressure and a second end;
a second tube having a first end and a second end terminating proximate a first nostril of a person;
a third tube having a first end and a second end terminating proximate a second nostril of a person;
a switch in communication with and interposed between said second end of said first tube and said first end of each of said second tube and said first end of said third tube;
said switch being positioned so as to permit a user, upon activation of said switch, to alternate a flow of said oxygen under pressure from said second end of said first tube as between said first end of said second tube and said first end of said third tube so that said oxygen under pressure exits only one of said second end of said second tube and said second end of said third tube.

4. A method for switching delivery of oxygen between two oxygen carrying tubes comprising the steps of:
providing a first tube having a first end and a second end;
providing a second tube having a first end and a second end;
wherein said first end of said tube receives oxygen under pressure;
providing a third tube having a first end and a second end;
providing a fourth tube having a first end and a second end;
providing a switch in communication with each of said second end of said first tube, said second end of said second tube, said first end of said third tube and said first end of said fourth tube;
wherein said second end of said third tube terminates proximate a first nostril of a person;
wherein said second end of said fourth tube terminates proximate a second nostril of said person;
wherein said switch is positioned so as to permit a user, upon activation of said switch, to alternate a flow of said oxygen under pressure from said second end of said first tube, as between said first end of said third tube and said first end of said second tube so that said oxygen under pressure exits only one of said second end of said third tube and said second end of said fourth tube;
receiving oxygen under pressure in said first end of said first tube; and
activating said switch and thereby causing said oxygen under pressure from said second end of said first tube to be received in the other of said first end of said third tube and said first end of said fourth tube.

5. The method of claim 4 further comprising the step of providing a timing device to track an amount of time between activation of said switch.

6. The method of claim 5 wherein said timing device is alarm-type.

7. The method of claim 5 wherein said timing device is adjustable.

8. The method of claim 5 wherein said timing device is coupled to said switch so that at a predetermined time interval, said switch is activated.
